(19) United States
(12) Patent Application Publication
(10) Pub. No.: US 2026/0363747 A1
(43) Pub. Date: Nov. 19, 2026

(54) Self-Countersinking Orthopedic Screws and Methods

(71) Applicant: GLW, Inc., Englewood Cliffs, NJ (US)

(72) Inventor: Anna Zastrozna, Englewood Cliffs, NJ (US)

(73) Assignee: GLW, Inc., Englewood Cliffs, NJ (US)

(21) Appl. No.: 19/745,735

(22) Filed: Jul. 17, 2026

Related U.S. Application Data
(63) Continuation of application No. 18/221,073, filed on Jul. 12, 2023, now abandoned.
(60) Provisional application No. 63/388,408, filed on Jul. 12, 2022.

(51) Int. Cl. A61B 17/86

(52) U.S. Cl. CPC A61B 17/8685; A61B 17/8605; A61B 17/866

(57) ABSTRACT

An orthopedic screw has a head portion defining a proximal end and defining a proximal thread extending around the longitudinal axis and having a first outer diameter, a tip portion defining a cutting edge and a distal end, a body extending between the proximal end and the distal end along the longitudinal axis and defining a shaft having a second outer diameter that is less than the first outer diameter, and a neck portion defining a taper extending between the first outer diameter and the second outer diameter and having an outer surface that defines first and second cutting flutes that cooperatively define a cutting surface, a cutting relief, and a material removal channel that is continuous with the proximal thread. The proximal end defines a proximal opening to the cavity. Methods of installing an orthopedic screw are also described.

### CROSS-REFERERENCE TO RELATED APPLICATION

This application is a continuation of U.S. application Ser. No. 18/221,073, which was filed on Jul. 12, 2023 and which claims priority to U.S. Provisional Application No. 63/388,408, filed on Jul. 12, 2022. The entire contents of each of these related applications are hereby incorporated by reference into this disclosure.

### FIELD

The disclosure relates to the field of medical devices. More particularly, the disclosure relates to orthopedic screws and methods of installing an orthopedic screw.

### BACKGROUND

Orthopedic screws, also referred to as bone screws, are implantable medical devices that are commonly used for fracture stabilization and fixation. Orthopedic screws can be used alone or in association with a bone plate. In some applications, it is desirable to minimize the extent to which an orthopedic screw extends beyond the surface of a bone or bone plate following installation. Indeed, it is often desirable to eliminate any such extension. Currently available orthopedic screws, however, require use of separate tools and additional steps to achieve this desired positioning of screws relative to the bone surface.

A need remains, therefore, for improved orthopedic screws and methods of installing an orthopedic screw.

### BRIEF SUMMARY OF SELECTED EXAMPLES

Various example orthopedic screws are described and illustrated herein.

An example orthopedic screw comprises a head portion defining a proximal end and defining a proximal thread extending around the longitudinal axis and having a first outer diameter, a tip portion defining a cutting edge and a distal end, a body extending between the proximal end and the distal end along the longitudinal axis and defining a shaft having a second outer diameter that is less than the first outer diameter, and a neck portion defining a taper extending between the first outer diameter and the second outer diameter and having an outer surface that defines first and second cutting flutes that cooperatively define a cutting surface, a cutting relief, and a material removal channel that is continuous with the proximal thread. The proximal end defines a proximal opening to the cavity.

Another example orthopedic screw comprises a head portion defining a proximal end and defining a proximal thread extending around the longitudinal axis and having a first outer diameter, a tip portion defining a cutting edge and a distal end, a body extending between the proximal end and the distal end along the longitudinal axis and defining a shaft having a second outer diameter that is less than the first outer diameter, and a neck portion defining a taper extending between the first outer diameter and the second outer diameter and having an outer surface that defines first and second cutting flutes that cooperatively define a cutting surface, a cutting relief, and a material removal channel that is continuous with the proximal thread; a metal inner core member defining the tip portion and the circumferential wall; and a polymeric outer body member disposed circumferentially around the inner core member and comprising the body. The proximal end defines a proximal opening to the cavity.

Another example orthopedic screw comprises a metal inner core member extending along the longitudinal axis of the orthopedic screw and defining the proximal end and the distal end; a polymeric outer body member disposed circumferentially around the inner core member; a head portion defining a proximal thread extending around the longitudinal axis and having a first outer diameter; a body extending between the proximal end and the distal end along the longitudinal axis and defining a shaft having a second outer diameter that is less than the first outer diameter; and a neck portion defining a taper extending between the first outer diameter and the second outer diameter and having an outer surface that defines first and second cutting flutes that cooperatively define a cutting surface, a cutting relief, and a material removal channel that is continuous with the proximal thread.

Various example methods of installing an orthopedic screw are also described.

An example method of installing an orthopedic screw comprises inserting a driver into a cavity of an orthopedic screw according to an embodiment until the driver contacts the transverse wall of the cavity, which is positioned axially beyond the distal end of the head portion of the orthopedic screw relative to the proximal end of the screw; and rotating the driver to advance the orthopedic screw into a bone. The rotating step is performed until the proximal surface of the orthopedic screw is positioned beneath the outer surface of the bone.

Another example method of installing an orthopedic screw comprises inserting the distal end of an orthopedic screw an orthopedic screw according to an embodiment through a passageway defined by a bone plate; inserting a driver into a cavity of the orthopedic screw until the driver contacts the transverse wall of the cavity, which is positioned axially beyond the distal end of the head portion of the screw relative to the proximal end of the screw; and rotating the driver to advance the orthopedic screw into a bone. The rotating step is performed until the proximal surface of the orthopedic screw is positioned beneath the upper surface of the bone plate relative to the outer surface of the bone.

Additional understanding of the claimed orthopedic screws and methods of installing an orthopedic screw can be obtained by reviewing the detailed description of selected examples, below, with reference to the appended drawings.

### DESCRIPTION OF FIGURES

FIG. 1 is a side view, partially broken away, of a prior art orthopedic screw extending through a bone plate and into a bone, each of which is shown in section.

FIG. 2 is a sectional view, partially broken away, of another prior art orthopedic screw extending through a bone plate and into a bone.

FIG. 3 is a side view, partially broken away, of another prior art orthopedic screw extending into a bone, which is shown in section.

FIG. 4 is a side view, partially broken away, of another prior art orthopedic screw extending through a washer and into a bone, each of which is shown in section.

FIG. 5 is a sectional view, partially broken away, of another prior art orthopedic screw extending through a bone plate and into a bone.

FIG. 6 is a perspective view, partially broken away, of an example orthopedic screw.

FIG. 7 is a sectional view, partially broken away, of the example orthopedic screw illustrated in FIG. 6 extending through a bone plate and into a bone.

FIG. 8 is a side view, partially broken away, of the example orthopedic screw illustrated in FIGS. 6 and 7 extending through a bone plate and into a bone, each of which is shown in section.

FIG. 9 is a perspective view, partially broken away, of another example bone screw.

FIG. 10 is a sectional view, partially broken away, of the example orthopedic screw illustrated in FIG. 9 extending through a bone plate and into a bone.

FIG. 11 is a side view, partially broken away, of the orthopedic screw illustrated in FIGS. 9 and 10 extending through a bone plate and into a bone, each of which is shown in section.

FIG. 12 is a sectional view, partially broken away, of the orthopedic screw illustrated in FIGS. 9 and 10 extending through a bone plant and into a bone.

FIG. 13 is a side view, partially broken away, of the example orthopedic screw illustrated in FIGS. 9 and 10 extending into a bone, which is shown in section.

FIG. 14 is a side view, partially broken away, of the example orthopedic screw illustrated in FIGS. 9 and 10 extending through a washer and into a bone, which is shown in section.

### DETAILED DESCRIPTION OF SELECTED EXAMPLES

The following detailed description and the appended drawings describe and illustrate various example orthopedic screws and methods of installing an orthopedic screw. The description and illustration of these examples enable one skilled in the art to make and use examples of the inventive orthopedic screws and to perform example methods of installing an orthopedic screw. The description and drawings exemplify the invention; they do not limit its scope or its protection.

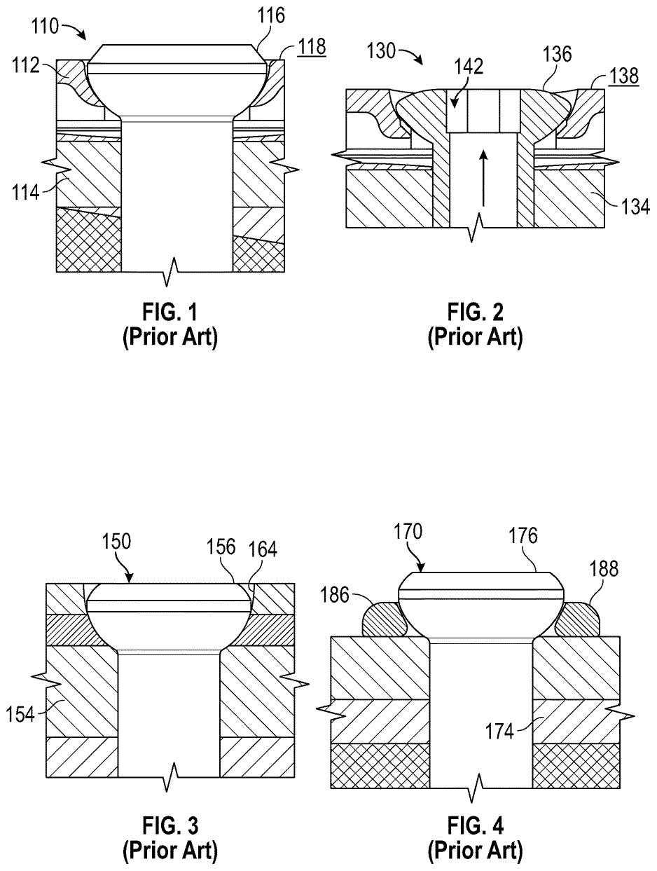

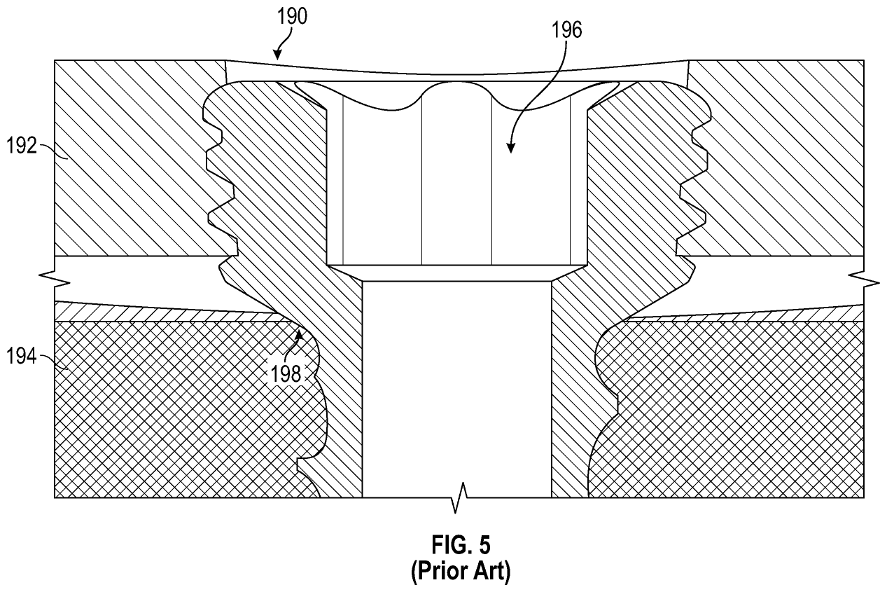

Each of FIGS. 1, 2, 3, 4, and 5 illustrates an orthopedic screw known in the art. FIG. 1 illustrates a headed orthopedic screw 110 extending through a bone plate 112 and into a bone 114. While fully seated in the bone plate 112 and bone 114, the head 116 of the orthopedic screw 110 remains prominent above the upper surface 118 of the bone plate 112. FIG. 2 illustrates a low-profile headed orthopedic screw 130. The low profile nature of the orthopedic screw 130 positions the head 136 of the orthopedic screw 130 flush with the upper surface 138 of the bone plate 132. The low profile nature of the orthopedic screw 130 also, however, provides limited volume in the head 136, necessitating a shallow driver recess 142 that can provide less than optimal driver engagement during installation of the orthopedic screw 130. FIG. 3 illustrates a headed orthopedic screw 150 installed directly into a bone 154 following use of a countersinking instrument and performance of a separate step during installation to remove bone to form a cavity 164 for receiving the head 156 of the orthopedic screw 150. FIG. 4 illustrates a headed orthopedic screw 170 installed through a washer 186 and into a bone 174. The head 176 of the orthopedic screw 170 remains prominent above the upper surface 188 of the washer 188. FIG. 5 illustrates a locking orthopedic screw 190 extending through a bone plate 192 and into a bone 194. The orthopedic screw 190 has a shallow driver recess 196 and a neck portion 198 that provides a mechanical stop that prevents the head of the orthopedic screw 190 from being countersunk into the bone plate 192 and bone 194 without the performance of additional steps and the use of additional instruments during implantation of the orthopedic screw 190.

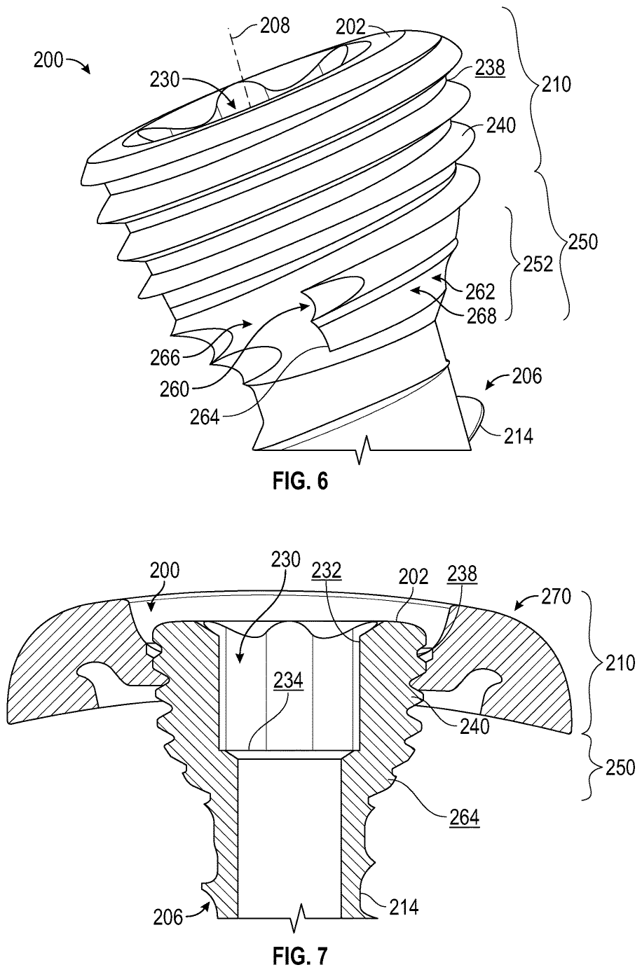

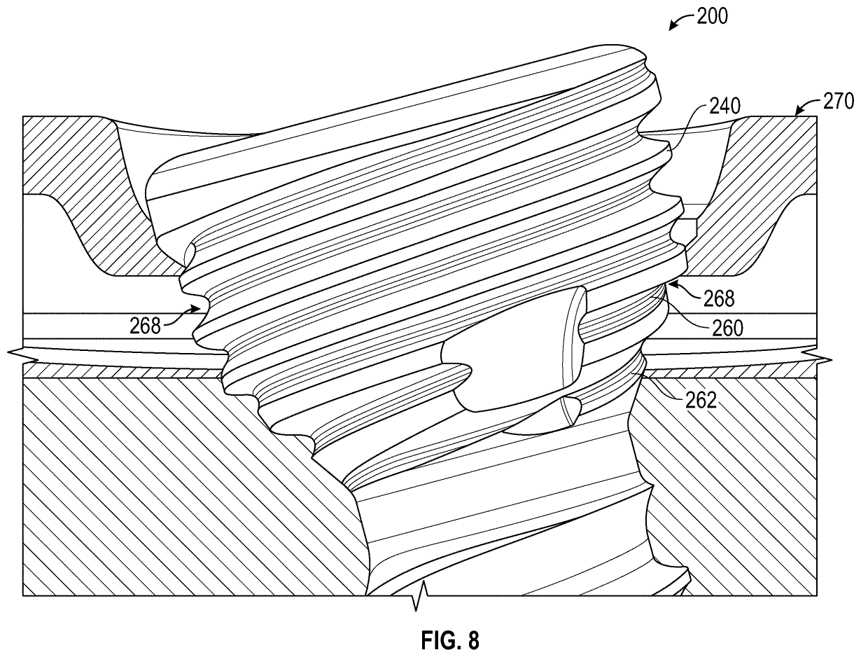

FIGS. 6, 7, and 8 illustrate a first example orthopedic screw 200. The orthopedic screw 200 is a locking screw with a threaded head for forming a locking engagement with threads of a bone plate, such as bone plate 270 illustrated in FIGS. 7 and 8. For simplicity, each of FIGS. 6,7, and 8 illustrates only a portion of the orthopedic screw 200.

The orthopedic screw 200 has a proximal end 202, a distal end (not visible in the Figures), and a body 206 extending between the proximal end 202 and the distal end 204 along a longitudinal axis 208. The orthopedic screw 200 includes a head portion 210 that terminates with the proximal end 202, a tip portion (not illustrated in the Figures) that terminates with the distal end, and a shaft 214 extending between the head portion 210 and the tip portion 212 and comprising the body 206. The tip portion provides structure for cutting into tissue, such as bone and/or cartilage, and the head portion 210 provides structure for interacting with one or more tools for placing or implanting the orthopedic screw 200 into tissue, such as a driver. In certain embodiments, the orthopedic screw 200 defines an inner lumen, allowing it to be passed over a separate member, such as a wire, to facilitate placement and/or positioning during implantation. Orthopedic screws according to other embodiments lack a full length lumen and comprise a solid body orthopedic screw.

The head portion 210 defines a recess 230 that is bounded by a circumferential wall 232 and a transverse wall 234. The circumferential wall 232 extends around the longitudinal axis 208 of the orthopedic screw 200. In cannulated embodiments, the transverse wall 234 defines an opening into the inner lumen.

The circumferential wall 232 defines structure that facilitates interaction with a tool, such as a driver, that can be used to drive the orthopedic screw 200 into, and remove the screw 200 from, tissue, such as cartilage and/or bone. The transverse wall 234 may also define structure that facilitates such interaction. As such, the circumferential wall 232, the transverse wall 234, and, as a result, the cavity 230 may have any suitable configuration and a skilled artisan will be able to select an appropriate configuration for each of these structures in an orthopedic screw according to a particular embodiment based on various considerations, including the configuration and nature of any driver with which the orthopedic screw is intended to be used. Examples of suitable configurations include conventional configurations for screw heads, including hex, star-shaped configurations, such as configurations compatible with TORX brand drivers, and other configurations. In the illustrated example, the head portion 210 is an enlarged structure relative to the tip portion and body 206 of the orthopedic screw 200.

In this embodiment, the external surface 238 of the head portion 210 defines head thread 240. In this example, the head thread 240 is oriented such that the head thread 240 engages and forms a locking engagement with threads of a bone plate, such as bone plate 270 illustrated in FIG. 7, through which the orthopedic screw 200 is disposed while being implanted upon application of a clockwise rotational force to the orthopedic screw 200.

In this embodiment, the head thread 240 extends from the proximal end 202 to the distal end of the head portion 210. A neck portion 250 defines a taper 252 from a first, relatively large outer diameter of the head portion 210 to a second, relatively small outer diameter of the shaft 214. The outer surface 252 of the neck portion 250 includes at least one set of dual adjacent cutting flutes 260, 262 that cooperatively define a cutting surface 264, a cutting relief 266, and a material removal channel 268. Additional sets of dual adjacent cutting flutes can be included in orthopedic screws according to embodiments. Suitable numbers of sets of dual adjacent cutting flutes that can be included include, but are not limited to, two sets of dual adjacent cutting flutes, three sets of dual adjacent cutting flutes, and more than three sets of dual adjacent cutting flutes. The material removal channel provides structure for removing material during implantation of orthopedic screw 200 while also providing an extension of the head thread 240, which facilitates formation of locking engagement of the orthopedic screw 200 with mating structure, such as structure defined by a bone plate. Furthermore, the material removal channel 268 provides a structural lead-in structure that enables head insertion into the bone by eliminating a mechanical stop that would otherwise be defined by neck portion 250. The material removal channel 268 can have any cross-sectional profile suitable for removal of material during implantation of the bone screw 200, such as a drill flute profile or a screw thread profile. During installation, the cutting flutes 260, 262 cut away bone to provide space in the bone to receive the head portion 210 of the orthopedic screw. As such, the orthopedic screw 200 can be countersunk into a bone or bone plate and a bone simply by installing the bone screw itself, eliminating the need for additional instruments and additional steps during installation.

The structural features of this embodiment allow orthopedic screw 200 to be used with or without a bone plate while being countersunk below the appropriate outermost surface of the bone or plate, as appropriate, without requiring the use of additional instruments or the performance of additional steps to achieve such positioning.

As best illustrated in FIG. 7, the inclusion of the cutting flutes 260, 262, and the need to position the cutting flutes 260, 262 radially outward of the relatively smaller outer diameter of the shaft 214, provides additional volume in the head portion 210 and neck portion 250 of the orthopedic screw 200. In the illustrated embodiment, the additional volume is used to for a cavity 230 having a depth that extends axially beyond the distal end of the head portion 210 of the orthopedic screw 200, and into the neck portion 250 of the orthopedic screw 200, which defines taper 252.

This structural configuration allows for a driver to be inserted to a greater depth during installation of the orthopedic screw, forming a more rugged engagement between the driver and the orthopedic screw 200, which is advantageous when driving the orthopedic screw 200 to a depth that provides the desired countersunk position relative to a bone or bone plate.

Also, as best illustrated in FIG. 8, the additional volume also increases engagement between head thread 240 and mating structure of bone plate 270 without interruption of cutting flutes 260, 262 in the bone plate to optimize cantilever strength between the orthopedic screw 200 and bone plate.

FIG. 8 illustrates the orthopedic screw 200 extending through bone plate 270 at a non-orthogonal angle. When inserted in this manner, as illustrated in the Figure, the material removal channel 268 act as an extension of the head thread 240, providing additional engagement between head thread 240 and mating structure of bone plate 270. This structural configuration allows for the establishment of a stable locking engagement between the orthopedic screw 200, bone plate 270 and bone even when the orthopedic screw is inserted at a non-orthogonal angle, as shown.

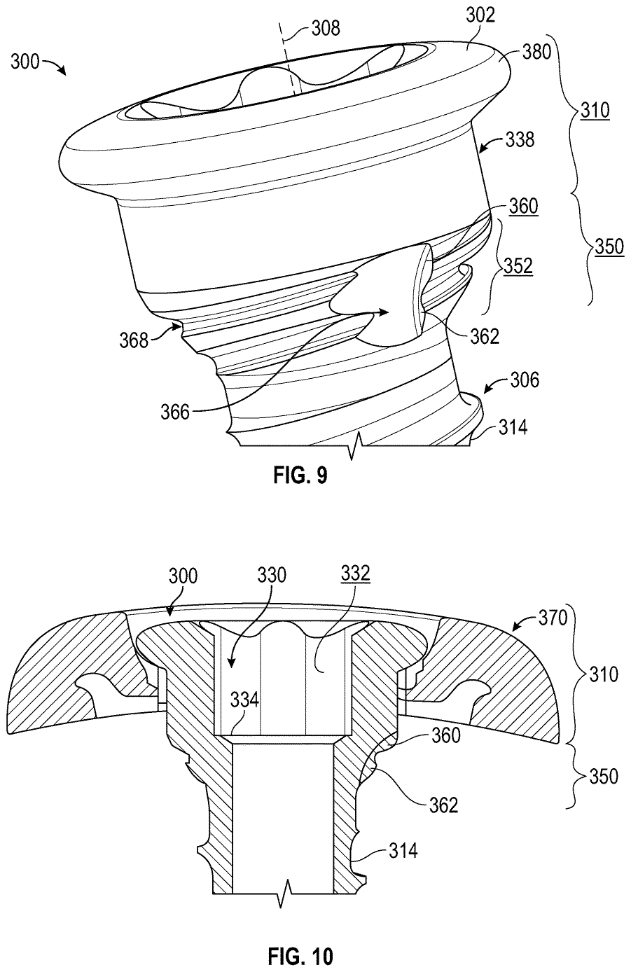

Each of FIGS. 9 and 10 illustrates a second example orthopedic screw 300. The orthopedic screw 300 is a non-locking screw with a non-threaded head and a circumferental flange for providing a mechanical stop to insertion into a bone plate, such as bone plate 370 illustrated in FIG. 10. For simplicity, each of FIGS. 9 and 10 illustrates only a portion of the orthopedic screw 300.

The orthopedic screw 300 has a proximal end 302, a distal end (not visible in the Figures), and a body 306 extending between the proximal end 302 and the distal end along a longitudinal axis 308. The orthopedic screw 300 includes a head portion 310 that defines a proximal flange 380 that forms the proximal end 302, a tip portion (not illustrated in the Figures) that terminates with the distal end, and a shaft 314 extending between the head portion 310 and the tip portion 312 and comprising the body 306. The tip portion 312 provides structure for cutting into tissue, such as bone and/or cartilage, and the head portion 310 provides structure for interacting with one or more tools for placing or implanting the orthopedic screw 300 into tissue, such as a driver.

The head portion 310 defines a recess 330 that is bounded by a circumferential wall 332 and a transverse wall 334. The circumferential wall 332 extends around the longitudinal axis 308 of the orthopedic screw 300.

The circumferential wall 332 defines structure that facilitates interaction with a tool, such as a driver, that can be used to drive the orthopedic screw 300 into, and remove the screw 300 from, tissue, such as cartilage and/or bone. The transverse wall 334 may also define structure that facilitates such interaction. As such, the circumferential wall 332, the transverse wall 334, and, as a result, the cavity 330 may have any suitable configuration and a skilled artisan will be able to select an appropriate configuration for each of these structures in an orthopedic screw according to a particular embodiment based on various considerations, including the configuration and nature of any driver with which the orthopedic screw is intended to be used. Examples of suitable configurations include conventional configurations for screw heads, including hex, star-shaped configurations, such as configurations compatible with TORX brand drivers, and other configurations. In the illustrated example, the head portion 310 is an enlarged structure relative to the tip portion and body 306 of the orthopedic screw 300.

In this embodiment, the external surface 338 of the head portion 310 is a flat surface, lacking a head thread.

A neck portion 350 defines a taper 352 from a first, relatively large outer diameter of the head portion 310 to a second, relatively small outer diameter of the shaft 314. The outer surface 352 of the neck portion 350 includes at least one set of dual adjacent cutting surfaces 360, 362, cutting relief 366, and a material removal channel 368. Additional sets of dual adjacent cutting flutes can be included in orthopedic screws according to embodiments. Suitable numbers of sets of dual adjacent cutting flutes that can be included include, but are not limited to, two sets of dual adjacent cutting flutes, three sets of dual adjacent cutting flutes, and more than three sets of dual adjacent cutting flutes. The material removal channel 368 is similar to the material removal channel 268 in orthopedic screw 200 described above and illustrated in FIGS. 6, 7, and 8, but is not continuous with a head thread in this example. During installation, the cutting surfaces 360, 362 cut away bone to provide space in the bone to receive the head portion 310 of the orthopedic screw 300. As such, the orthopedic screw 300 can be countersunk into a bone or bone plate and a bone simply by installing the bone screw itself, eliminating the need for additional instruments and additional steps during installation.

As best illustrated in FIG. 10, the inclusion of the cutting flutes 360, 362, and the need to position the cutting flutes 360, 362 radially outward of the relatively smaller outer diameter of the shaft 314 provides additional volume in the head portion 310 and neck portion 350 of the orthopedic screw 300. In the illustrated embodiment, the additional volume is used to for a cavity 330 having a depth that extends axially beyond the distal end of the head portion 310 of the orthopedic screw 300, and into the neck portion 350 of the orthopedic screw 300. This structural configuration allows for a driver to be inserted to a greater depth during installation of the orthopedic screw, forming a more rugged engagement between the driver and the orthopedic screw 300, which is advantageous when driving the orthopedic screw 300 to a depth that provides the desired countersunk position relative to a bone or bone plate.

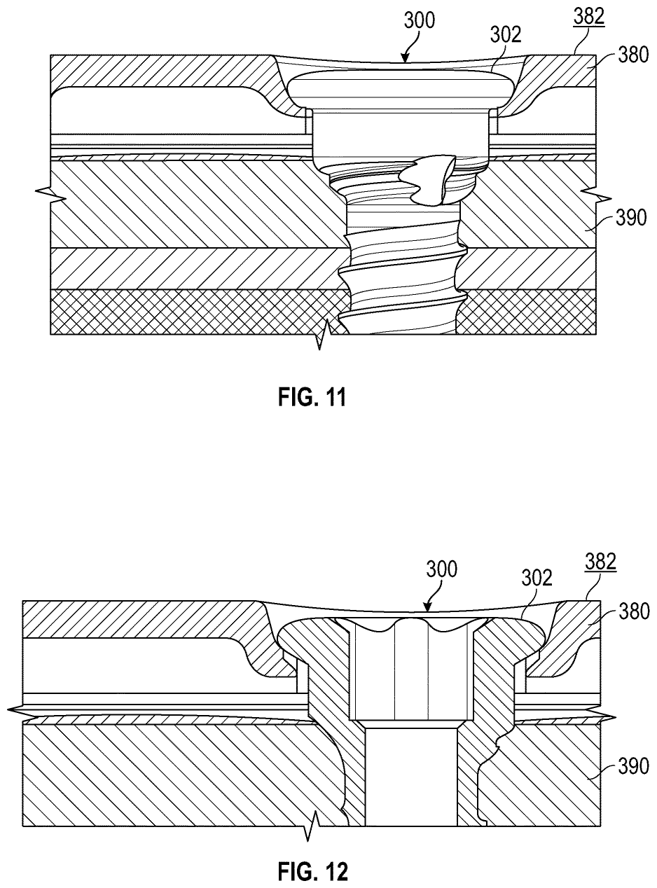

Each of FIGS. 11 and 12 illustrate the orthopedic screw 300 extending through a bone plate 380 and into a bone 390. Each Figure shows the countersunk position that can be achieved, with the proximal end 302 of the orthopedic screw 300 disposed beneath the upper surface 382 of the bone plate 380 relative to the bone 390.

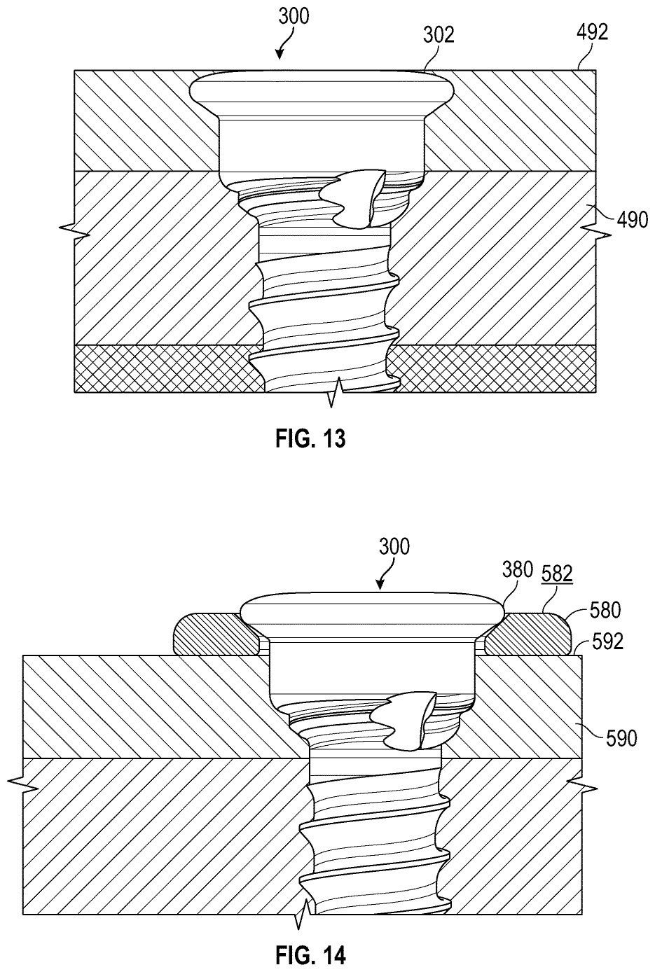

FIG. 13 illustrates the orthopedic screw 300 extending directly into a bone 490. The Figure shows the countersunk position that can be achieved, with the proximal end 302 of the orthopedic screw 300 disposed beneath the outer surface 492 of the bone 490.

FIG. 14 illustrates the orthopedic screw 300 extending through a washer 580 and into a bone 590. The orthopedic screw 300 has a low profile relative to the outer surface 592 of the bone 590, with only a portion of the proximal flange 380 disposed above the upper surface 582 of the washer 580 relative to the bone 590.

In all embodiments, the orthopedic screw can be made of any material suitable for use in medical devices intended for orthopedic use, including use as a long-term implant. Examples of suitable materials include metals, metal alloys, and polymeric materials. Examples of suitable metals include, but are not limited to, Titanium, Magnesium, and other metals. Examples of suitable metal alloys include, but are not limited to, Ti6Al4V, 316 LVM, 1.4441Ti-13Nb-13Zr, Ti-12Mo-6Zr-2Fe, Ti-15Mo-5Zr-3Al, Ti-15Mo, Ti-35Nb-7Zr-5Ta and Ti-29Nb-13Ta-4.6Zr Ti-6Al-7Nb and Ti-15Sn-4Nb-2Ta-0.2Pd Co—Cr—Mo alloys. Examples of suitable polymeric materials include, but are not limited to, polyaryletherketone (PAEK), polyether ether ketone (PEEK), PEEK (90G, 450G, I2, I4), Polyamide, PA66, carbon fiber reinforced polyaryletherketone (CFR PAEK), polyether ketone ketone (PEKK), carbon fiber reinforced polyether ketone ketone (CFR PEKK), carbon fiber reinforced polyether ether ketone (CFR PEEK), CFR PEEK (90G CA30, 90G CA20, 450G CA30, 450G CA20, I2 CF20, I2 CF30, I4 CF30, I4 CF20), Polyamide CFR, and PA66 CFR.

Orthopedic screws according to some embodiments include multiple components. In these embodiments, the components can be formed of the same or different materials. For example, an orthopedic screw according to an embodiment can include an inner core member that defines a distal tip, head core, and a series of cutting teeth on the head core as described above, and an outer body member that circumferentially surrounds the inner core member and defines a distal thread and a head thread. In this embodiment, it is considered advantageous to include an inner core member formed of a first metallic material, such as a metal or a metal alloy, and an outer body member formed of a second, different material, such as a polymeric material, a blended material such as a carbon fiber reinforced polymer, or another non-metallic material. It is considered particularly advantageous to include an inner core member formed of a Titanium alloy, such as Ti6Al4V, and an outer body member formed of a second, different material, such as CFR PEEK at least because this combination of materials provides desirable characteristics and a favorable balance between manufacturability and strength considerations. In these embodiments, the outer body member can be made of any material suitable for use in medical devices intended for orthopedic use, including use as a long-term implant. Examples of suitable types of materials include, but are not limited to, polymeric materials, blended materials such as carbon fiber reinforced polymers, and other materials. Examples of suitable polymeric materials include, but are not limited to, PAEK, CFR PAEK, PEKK, CFR PEKK, PEEK, CFR-PEEK, PEEK (90G, 450G, I2, I4), Polyamide, and PA66. Examples of suitable blended materials include, but are not limited to, PEEK-Carbon materials, CFR PAEK, CFR PEKK, CFR PEEK (90G CA30, 90G CA20, 450G CA30, 450G CA20, I2 CF20, I2 CF30, I4 CF30, I4 CF20), Polyamide CFR, PA66 CFR.

It is noted that the materials used in an orthopedic screw according to a particular embodiment can include additives, coatings, fillers, and/or other elements if desired. For example, antibiotics, bioactive glass, silver, copper, or another material that can reduce bacterial colonization of the orthopedic screw following implantation can be included in the material of the inner core member, the outer body member, or both.

The structural features of orthopedic screws according to embodiments provide several advantages over prior art orthopedic screws, including elimination of a separate countersinking step during installation and elimination of the need for a separate instrument for achieving a countersunk positioning; and enablement of the use of thinner bone plates, as the self-countersinking features cut into the bone and ensures the screw head fully seats flush with or beneath the upper surface of the plate and ensures full engagement of the locking threads to provide greater cantilever strength between the screw and plate than that provided by known orthopedic screws.

The self-countersinking structural arrangement on a non-locking head in an orthopedic screw according to an embodiment enables the use of relatively thin bone plates in comparison to bone plates suitable for use with conventional non-locking orthopedic screws, as the self-countersinking features cut into the bone and ensures the screw head to fully seats flush with or beneath the upper surface of the plate and ensures the plate to be drawn to the bone.

Also, the self-countersinking structural arrangement on a head in an orthopedic screw according to an embodiment provides volume for a relatively deeper driver cavity in comparison to heads of conventional orthopedic screws, which can enable more secure insertion of the orthopedic screw by providing reduced potential for stripping of the socket defined by the cavity, which is a known issue with conventional low profile orthopedic screws.

Also, the self-countersinking structural arrangement on a head in an orthopedic screw according to an embodiment provides provides for the removal of bone around the head during insertion of the orthopedic screw that could otherwise contribute to interference, bone stress, and, possibly, bone fracture.

Methods of installing an orthopedic screw are also provided. An example method comprises inserting a driver into a cavity of an orthopedic screw according to an embodiment, such as orthopedic screw 200 or orthopedic screw 300. This is performed until the driver contacts the transverse wall of the cavity, which is positioned axially beyond the distal end of the head portion of the screw relative to the proximal end of the screw. Another step comprises rotating the driver to advance the orthopedic screw into a bone. This is performed until the proximal surface of the orthopedic screw is positioned beneath the outer surface of the bone.

Another example method comprises inserting the distal end of an orthopedic screw an orthopedic screw according to an embodiment, such as orthopedic screw 200 or orthopedic screw 300, through a passageway defined by a bone plate. Another step comprises inserting a driver into a cavity of the orthopedic screw. This is performed until the driver contacts the transverse wall of the cavity, which is positioned axially beyond the distal end of the head portion of the screw relative to the proximal end of the screw. Another step comprises rotating the driver to advance the orthopedic screw into a bone. This is performed until the proximal surface of the orthopedic screw is positioned beneath the upper surface of the bone plate relative to the outer surface of the bone.

Specific contemplated example orthopedic screws include, but are not limited to, the following:

An orthopedic screw with self-countersinking features at a neck region along a taper that transitions from a relatively large outer diameter of the head to a relatively small outer diameter of the shaft.

An orthopedic screw with locking thread along the screw head and self-countersinking features at a neck region along a taper that transitions from a relatively large outer diameter of the head to a relatively small outer diameter of the shaft.

A non-locking orthopedic screw according to an embodiment and a bone plate, in combination, where an opening in the plate allows full bone screw head insertion, a narrowing towards the underside of the plate retains screw head in plate, a self-countersinking part of the screw extends below the plate cutting into the bone, and self-countersinking part of the screw and socket recess extends below the plate for sufficient driver engagement.

A locking orthopedic screw according to an embodiment and a bone plate, in combination where an opening in the plate allows full bone screw head insertion, a narrowing towards the underside of the plate retains screw head in plate, a self-countersinking part of the screw extends below the plate cutting into the bone, and a self-countersinking part of the screw and socket recess extends below the plate for sufficient driver engagement.

An orthopedic screw according to an embodiment and a washer, in combination, where the washer retains the upper portion of the screw head above the bone and the self-countersinking part of the screw extends below the washer cutting into the bone.

The particular examples disclosed herein have been selected by the inventor simply to describe and illustrate examples of the invention and are not intended to limit the scope of the invention or its protection, which is to be given the full breadth of the appended claims and any and all equivalents thereof. Artisans of ordinary skill will appreciate that various modifications and alternatives for the described and illustrated examples can be developed in light of the overall teachings of the disclosure. These modifications and alternatives are within the scope of the invention. For example, an element or elements of one example orthopedic screw described and illustrated herein can be combined with an element or elements of another example orthopedic screw without departing from the scope of the invention. Similarly, a step or steps of one example method described and illustrated herein can be combined with a step or steps of another example without departing from the scope of the invention.

We claim:

1. An orthopedic screw having a longitudinal axis, the orthopedic screw comprising:
a head portion defining a proximal end and having and external surface and a first outer diameter;
a tip defining a distal end;
a body extending between the proximal end and the distal end along the longitudinal axis, the body defining a shaft having a second outer diameter that is less than the first outer diameter; and
a neck portion extending between the first outer diameter and the second outer diameter, the neck portion having an outer surface defining at least one set of dual adjacent cutting flutes that cooperatively define a cutting surface, a cutting relief, and a material removal channel.

2. The orthopedic screw of claim 1, wherein the at least one set of dual adjacent cutting flutes comprises two sets of dual adjacent cutting flutes.

3. The orthopedic screw of claim 1, wherein the external surface defines a head thread; and
wherein the material removal channel comprises an extension of the head thread that provides structure for removing material during implantation of the orthopedic screw.

4. The orthopedic screw of claim 3, wherein the extension of the head thread facilitates formation of locking engagement of said orthopedic screw with a mating structure.

5. The orthopedic screw of claim 4, wherein the mating structure is defined by a bone plate.

6. The orthopedic screw of claim 1, wherein the at least one set of dual adjacent cutting flutes extends radially outward of the second outer diameter.

7. The orthopedic screw of claim 1, wherein the neck portion defines a taper that extends between the first outer diameter and the second outer diameter.

8. The orthopedic screw of claim 7, wherein the head portion defines a cavity, a circumferential wall, and a transverse wall; and
wherein the cavity is bounded by the circumferential wall and the transverse wall.

9. The orthopedic screw of claim 8, wherein the proximal end defines a proximal opening to the cavity.

10. The orthopedic screw of claim 9, wherein the cavity extends into the neck portion.

11. The orthopedic screw of claim 1, wherein the head portion defines a continuous circumferential flange that forms the proximal end.

12. The orthopedic screw of claim 1, wherein the proximal end has a third outer diameter that is different from the first outer diameter and the second outer diameter; and
wherein the third outer diameter is less than the first outer diameter and greater than the second outer diameter.

13. An orthopedic screw having a longitudinal axis, the orthopedic screw comprising:
a head portion defining a proximal end and having a first outer diameter and an external surface defining a head thread;
a tip defining a distal end;
a body extending between the proximal end and the distal end along the longitudinal axis, the body defining a shaft having a second outer diameter that is less than the first outer diameter; and
a neck portion extending between the first outer diameter and the second outer diameter, the neck portion having an outer surface defining at least one set of dual adjacent cutting flutes defining a cutting surface, a cutting relief, and a material removal channel comprising an extension of the head thread.

14. The orthopedic screw of claim 13, wherein the neck portion defines a taper that extends between the first outer diameter and the second outer diameter.

15. The orthopedic screw of claim 14, wherein the head portion defines a cavity, a circumferential wall, and a transverse wall; and
wherein the cavity is bounded by the circumferential wall and the transverse wall.

16. The orthopedic screw of claim 15, wherein the proximal end defines a proximal opening to the cavity.

17. The orthopedic screw of claim 16, wherein the cavity extends axially into the neck portion.

18. The orthopedic screw of claim 13, wherein the proximal end has a third outer diameter that is different from the first outer diameter and the second outer diameter; and
wherein the third outer diameter is less than the first outer diameter and greater than the second outer diameter.

19. An orthopedic screw having a longitudinal axis, the orthopedic screw comprising:
a head portion having a first outer diameter and defining a proximal end, a cavity, an opening to the cavity, and an external head thread;
a tip defining a distal end;
a body extending between the proximal end and the distal end along the longitudinal axis, the body defining a shaft having a second outer diameter that is less than the first outer diameter; and
a tapered neck portion extending between the head portion and the body, the neck portion having an outer surface defining at least one set of dual adjacent cutting flutes defining a cutting surface, a cutting relief, and a material removal channel comprising an extension of the head thread;
wherein the cavity extends axially into the neck portion.

20. The orthopedic screw of claim 19, wherein the proximal end has a third outer diameter that is different from the first outer diameter and the second outer diameter; and
wherein the third outer diameter is less than the first outer diameter and greater than the second outer diameter.
